(19) United States
(12) Patent Application Publication
(10) Pub. No.: US 2008/0300679 A1
(43) Pub. Date: Dec. 4, 2008

(54) Diffractive Intraocular Lens

(76) Inventor: Griffith E. Altmann, Pittsford, NY (US)

Correspondence Address: Bausch & Lomb Incorporated, One Bausch & Lomb Place, Rochester, NY, 14604-2701, US

(21) Appl. No.: 11/756,973

(22) Filed: Jun. 1, 2007

(51) Int. Cl. A61F 2/16

(52) U.S. Cl. 623/6.31

(57) ABSTRACT

An IOL, comprising a first diffractive region, and a second diffractive region having a power of a different magnitude than the first diffractive region, the first region adapted to diffract substantially all light at a first selected wavelength projected therethrough into a single order of diffraction, and the second region adapted to diffract substantially all light at a second selected wavelength projected therethrough into a single order of diffraction.

### FIELD OF THE INVENTION

The present invention relates to diffractive intraocular lenses (IOLs), and more particularly to diffractive multifocal IOLs.

### BACKGROUND OF THE INVENTION

IOLs are artificial lenses used to replace natural lenses of patients when their natural lenses are diseased or otherwise impaired. Under some circumstances a natural lens may remain in a patient's eye together with an implanted IOL. IOLs may be placed in either the posterior chamber or the anterior chamber of an eye.

IOLs typically have some optical power. IOLs to be placed in the posterior chamber typically have sufficient power to compensate for the removal of the natural lens. Lenses to be placed in the anterior chamber may supplement the power of the natural lens or may have power to compensate for removal of natural lens. IOLs come in a variety of configurations and materials.

Some IOLs are multifocal. Multifocal IOLs are IOLs that have at least two different optical powers. Typically, such multifocal IOLs ameliorate the effects of presbyopia by providing a wearer with optical power to permit near vision and distance vision. Multifocal IOLs where at least one of the powers is at least partially provided using a diffractive surface offer an advantage of being relatively small in profile compared to IOLs that are solely refractive. Therefore, diffractive IOLs are typically more easily injected into a patient's eye through a smaller incision.

FIG. 1 illustrates a conventional multifocal IOL 10 in which a curved surface 12 has a diffractive component 14 disposed thereon that is configured such that a first portion of the light incident on the diffractive component is diffracted and focused at a first focal distance (i.e., the first portion of light is directed into the first diffractive order of the diffractive component). The first focal distance of the first portion is determined by the combined optical powers of the diffractive component and the curved (i.e., refractive) surface.

A second portion of the light passes through the diffractive component without being diffracted (i.e., the light is directed into the zeroth diffractive order of the diffractive component). The second portion of light passes through the diffractive component of the lens without deviation due to diffraction. However, the light in the zeroth order is refracted due to the curvature of surface so as to be focused at a second focal distance.

FIG. 2 illustrates a second conventional multifocal IOL 20 in which a first region 22 of one surface of the IOL has a diffractive component 24. The first region of surface is configured such that a first portion of light is projected into the first order of the diffractive component which, in combination with the refractive power of surface, provides optical power sufficient for near vision. Light passing through the first region in the zeroth order of the diffractive portion is only refracted and combines with light from the outer portion 26 of the lens for distance vision. Accordingly, light in the first diffractive order is focused at a first distance and light in the zeroth diffractive order is focused at a second distance.

### SUMMARY

A disadvantage of diffractive multifocal IOLs that rely on refraction of a zeroth order is that lens thickness is relatively great due to the need for a suitably curved refractive surface to focus light in the zeroth order. Another disadvantage of focusing a zeroth order of diffraction is that light that is only refracted (i.e., undiffracted light in the zeroth order) has a relatively large amount of positive chromatic aberration compared to light that is at least partially diffracted; accordingly, a positively-powered refractive surface of the IOL that passes light in the zeroth order exacerbates positive chromatic aberration provided by a patient's cornea and/or natural lens.

Aspects of the present invention are directed to an IOL having at least two regions having diffractive power of different magnitudes than one another, each of the regions having substantially only a single non-zero order of diffraction (i.e., substantially no zeroth order diffraction) to provide focusing power for light incident on the lens.

Accordingly, substantially all of the light passing through a given region of the lens is diffracted into a corresponding single diffraction order.

Furthermore, in some embodiments, the two or more regions cover an entire or substantially an entire lens surface. It will be appreciated that in such embodiments, none or substantially none of the light passes through the lens undiffracted. In some embodiments, diffractive surfaces may be disposed on only a portion of the anterior surface or on only a portion of the posterior surface; however, the portions are arranged such that none of the light passes through the lens undiffracted.

A first aspect of the invention is directed to an IOL, comprising a first diffractive region, and a second diffractive region having a power of a different magnitude than the first diffractive region. The first region is adapted to diffract substantially all light at a first selected wavelength projected therethrough into a single non-zero order of diffraction, and the second region is adapted to diffract substantially all light at a second selected wavelength projected therethrough into a single non-zero order of diffraction.

In some embodiments, the first selected wavelength and the second selected wavelength are at substantially a same wavelength in the visible spectrum (i.e., within 100 nm of one another). In some embodiments, the first selected wavelength and the second selected wavelength are at a same wavelength in the visible spectrum (e.g., approximately 555 nm).

In some embodiments, the IOL further comprises a third diffractive region having a power of different magnitude than the first and the second regions. The third region is adapted to diffract substantially all light (at a selected wavelength) projected therethrough into a corresponding single order of diffraction.

In some embodiments, the first region and the second region project light into respective first orders of diffraction. The first and second regions may be disposed concentrically. The IOL may comprise one or more haptics. In some embodiments, the IOL is made of a material comprising at least one of silicone, PMMA, foldable hydrophilic acrylic and foldable hydrophobic acrylic. In some embodiments, the IOL further comprises a third region, concentric with the first and second regions, wherein the first region, the second region, and the third region form alternating regions of near and far focus.

Another aspect of the invention is directed to a method, comprising steps of (1) projecting light onto a first diffractive region and a second diffractive region of an IOL, the first region having a first diffractive optical power and the second region having a second diffractive optical power different of a different magnitude than the first diffractive optical power; and (2) diffracting substantially all light of a first selected wavelength projected onto the first region into a single order of diffraction; and (3) diffracting substantially all light of a second selected wavelength projected onto the second region into a single order of diffraction.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a schematic, cross-sectional illustration of a conventional multifocal diffractive IOL;

FIG. 2 illustrates another conventional multifocal, diffractive IOL;

FIG. 3A is a cross sectional view of an example of an embodiment of an IOL constructed according to aspects of the present invention; and

FIG. 3B is a plan view of the lens in FIG. 3A.

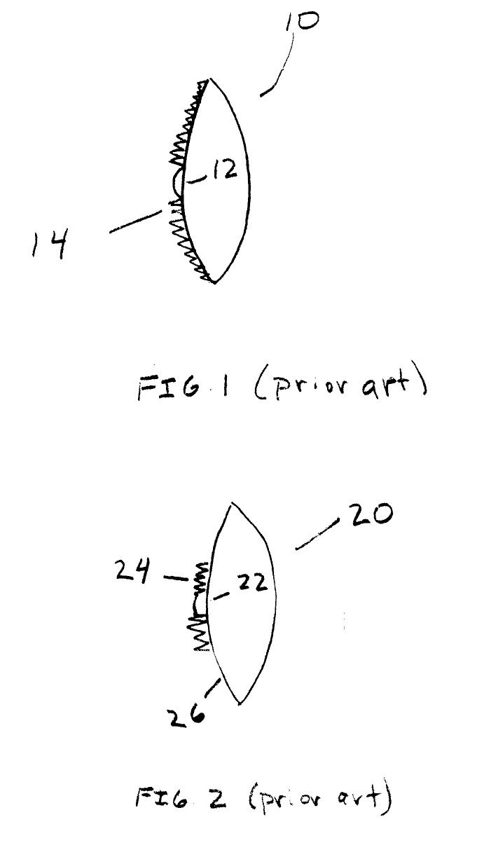

### DETAILED DESCRIPTION

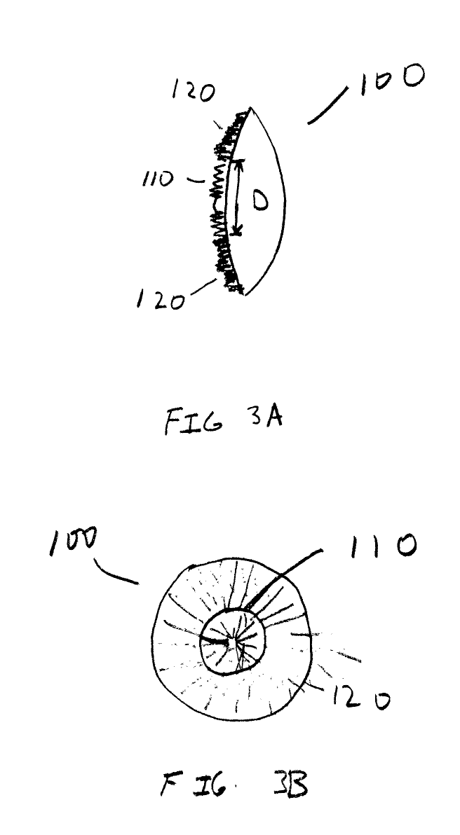

FIG. 3A is a cross sectional view of an example of an embodiment of an IOL 100 constructed according to aspects of the present invention.

IOL 100 includes a first diffractive region 110 having a diameter D, and a second diffractive region 120. First and second diffractive regions have powers of different magnitudes than one another and therefore have different focal points. The powers are selected according to conventional techniques to provide a patient vision at two different distances. For example, a first power may provide the patient with near vision for reading and a second power may be selected for distance vision. Although the illustrated lens has two regions having powers of different magnitude, any suitable number of regions may be provided (e.g., three, four or more regions). Typically when three or more regions having different powers are present, a first region provides near vision, a second provides distance vision and the remaining regions provide intermediate vision at one or more distances.

According to aspects of the invention, the first region is adapted to diffract substantially all light of a first selected wavelength projected therethrough into a single non-zero order of diffraction, and the second region is adapted to diffract substantially all light of a second selected wavelength projected therethrough into a single non-zero order of diffraction.

It will be appreciated that a lens so adapted can be constructed to be thinner than a lens adapted to project light into the zeroth order because there is no need to refract light in the zeroth order (i.e., any refractive power that may be provided is reduced due to the power provided by the diffractive surface). Typically, the non-zeroth order will be the first positive or the first negative order due to the ease of manufacture and/or efficiency with which diffraction into a first order can be achieved. However, according to aspects of the present invention, any positive or negative order of diffraction can be used to provide different powers in the regions. Diffractive regions can be formed on the anterior and/or posterior surfaces of an IOL.

In some embodiments, the first selected wavelength and the second selected wavelength are a same wavelength in the visible spectrum or substantially a same wavelength in the visible spectrum (e.g., within 20 nm of one another); however the first selected wavelength may be different than the second selected wavelength. In some embodiments in which the first selected wavelength is the same as the second selected wavelength, the wavelength is located at a wavelength of high ocular sensitivity (e.g., approximately 555 nm). Even if the first and second selected wavelength are different than one another, in some embodiments both wavelengths are located at wavelengths of high ocular sensitivity (e.g., the first selected wavelength is 555 nm and the second selected wavelength is 548 nm).

It will be appreciated that since all of the light used to form images using IOLs according to aspects of the present invention is formed at least in part by a diffractive surface, the IOL contributes a reduced amount of chromatic aberration compared to a diffractive lens that passes light through a zeroth diffraction order. In some embodiments, the lens transmits light having a reduced amount of positive chromatic aberration, and in some embodiments, the lens transmits light having negative chromatic aberration and as such is better able to compensate for positive chromatic aberration introduced by the patient's cornea, natural lens and/or other ocular media.

The term “adapted to project substantially all light at a selected wavelength projected therethrough into a single order of diffraction” means that of the light at the selected wavelength incident on a given location within a region of a lens, at least 90% of the light enters into the selected single diffraction order of that region. It will be appreciated that diffraction in a given region typically occurs with some coupling into orders other than the selected order. While a certain amount of such loss of light may be tolerated, higher transmission efficiency is typically desirable. For example, higher transmission efficiency will result in better low light vision and better contrast sensitivity. In some embodiments, at least 95% or more of the light is directed into a selected order.

Equation 1 is an equation providing a relationship between the grating element spacing and diffractive focal length for a given wavelength.

r_{q}=sqrt(2*q*λ*d) Equation 1

where
q=zone number
r_{q}=radius (i.e., spacing) of a concentric annular zone
λ=design wavelength
d=focal length of diffractive element

As can be seen in the equation, the spacing of the zones, r, is proportional to the focal length, d. Since power is inversely related to focal length, zone spacing decreases to achieve greater diffractive power.

Equation 2 is an equation providing a relationship between the grating depth and the transmission efficiency for a given order of diffracted light for a given wavelength.

I=sinc²(m−α) Equation 2

where
m=diffractive order
α=(n′−n)*Δ/λ
n′=refractive index of IOL material
n=refractive index of surrounding material (i.e., aqueous humor)
Δ=maximum depth of grating

In some embodiments of the present invention, a diffractive region is designed such that peak diffraction efficiency occurs for light at a wavelength at which maximum sensitivity of an eye occurs (i.e., approximately 555 nm), and as a result, light having shorter or longer wavelengths is less efficiently diffracted. An advantage of IOL's according to such aspects is that light having wavelengths substantially shorter than 555 nm (e.g., blue light, violet light and/or ultraviolet light) is at least partially out of focus on the eye. Blue light, violet light and UV light have been associated with age-related macular degeneration. It is believed that defocused blue, violet and UV light are less damaging to a person's macula (i.e., retina), because defocused energy has reduced intensity.

It is to be appreciated that the magnitude of defocus, as a function of wavelength, that can be achieved with a diffractive region is greater than can be typically achieved due to longitudinal chromatic aberration in a refractive IOL. For example, a typical diffractive lens may provide an optical power difference of 1 diopter (positive chromatic aberration) between violet light (400 nm) and green light (555 nm), while a diffractive element may provide an optical power difference of 10 diopters (negative chromatic aberration) between violet light and green light.

Furthermore, unlike blue-blocking chromophores which are commonly added to IOLs to reduce or eliminate blue light incident on a patient's retina, with IOLs according to aspects of the present invention, blue light still reaches a patient's retina. Blue light, even if it is defocused, reaching a patient's retina is believed to provide some benefits related to seasonal disorders.

FIG. 3B is a plan view of the lens 100 in FIG. 3A. It will be appreciated that the regions 110 and 120 of the lens are disposed concentrically. However, any suitable arrangement of the regions maybe be used. Also, although two concentrically disposed regions are shown, three, four or more concentrically disposed regions may be used in some embodiments. For example, the regions may be disposed as concentric regions, where alternate regions provide near and far focus.

Lenses according to aspects of the present invention may be configured for placement in any internal portion of a patient's eye (e.g., an anterior chamber or a posterior chamber). Although lens 100 is shown without haptics, one or more haptics may be added to facilitate positioning of an IOL in an eye. The haptics may be of any suitable shape (e.g., filament-shaped or plate-shaped). Lenses according to aspects of the invention may be made using any suitable manufacturing technique. However one suitable technique for forming the fine diffractive features is injection molding.

Lenses according to aspects of the invention may be made using any suitable material. For example, at least one of silicone, polymethyl methacrylate (PMMA), foldable hydrophilic acrylic or foldable hydrophobic acrylic acrylic may be used.

In the illustrated embodiment, light passing through all portions of the lens is diffracted. It is to be appreciated that although in the illustrated embodiment light passing through all portion of the lens will be diffracted, in any given embodiment of a lens, only portions of the lens through which light will pass, when the lens is in an eye provide advantages for chromatic aberration. Other portions of the lens (i.e., the outer periphery of the lens) may or may not be configured to diffract light.

The following is an example of a design of a 20-diopter “central add” bifocal, diffractive IOL made of silicone according to aspects of the present invention. Due to the use of diffractive power, although the lens is a 20 diopter lens, the base lens design has 15 diopters of refractive power, so it is thinner than a standard 20-diopter lens. Although the present lens is shaped to provide refractive power, in some embodiments, all optical power is provided diffractively.

The tables below show the optical prescriptions of the base lens and a diffraction grating disposed on the anterior surface of the exemple lens.

Zones 1-7 correspond to the central add region and have a diffractive power of 8.75 diopters and a total optical power of 23.75 diopters. Zones 8-40 correspond to the outer annular region and have a diffractive power of 5 diopters and a total optical power of 20 diopters. Hence the central add power is 3.75 diopters. The diameter of the central region is 1.9 mm.

Each of the first region (including zones 1-7) and the second region (including zones 8-40) are adapted to project substantially all light projected therethrough substantially into a corresponding single order of diffraction.

| | | |
|---|---|---|
| Design Wavelength | 555 | nm |
| IOL Material Refractive Index for Design Wavelength | 1.427 | |
| Aqueous Humor Refractive Index for Design | 1.336 | |
| Wavelength | | |
| Anterior Surface Radius | 12.0996 | |
| Posterior Surface Radius | −12.0996 | |
| Optic Zone Diameter | 6 | mm |
| Optic Body Diameter | 6 | mm |
| Central Thickness | 1.056 | mm |
| Edge Thickness | 0.3 | mm |
| Grating Depth | 6.099 | μm |

| Zone number | Radius |
|---|---|
| 1 | 0.3562 |
| 2 | 0.5037 |
| 3 | 0.6169 |
| 4 | 0.7123 |
| 5 | 0.7964 |
| 6 | 0.8724 |
| 7 | 0.9423 |
| 5 | 1.0536 |
| 6 | 1.1541 |
| 7 | 1.2466 |
| 8 | 1.3327 |
| 9 | 1.4135 |
| 10 | 1.4900 |
| 11 | 1.5627 |
| 12 | 1.6322 |
| 13 | 1.6988 |
| 14 | 1.7630 |
| 15 | 1.8248 |
| 16 | 1.8847 |
| 17 | 1.9427 |
| 18 | 1.9990 |
| 19 | 2.0538 |
| 20 | 2.1071 |
| 21 | 2.1592 |
| 22 | 2.2100 |
| 23 | 2.2596 |
| 24 | 2.3082 |
| 25 | 2.3558 |
| 26 | 2.4025 |
| 27 | 2.4483 |
| 28 | 2.4932 |
| 29 | 2.5373 |
| 30 | 2.5807 |
| 31 | 2.6234 |
| 32 | 2.6653 |
| 33 | 2.7067 |
| 34 | 2.7474 |
| 35 | 2.7875 |
| 36 | 2.8270 |
| 37 | 2.8660 |
| 38 | 2.9045 |
| 39 | 2.9424 |
| 40 | 2.9799 |

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

1. An IOL, comprising:
a first diffractive region; and
a second diffractive region having a power of a different magnitude than the first diffractive region, the first region adapted to diffract substantially all light at a first selected wavelength projected therethrough into a single non-zero order of diffraction, and the second region adapted to diffract substantially all light at a second selected wavelength projected therethrough into a single non-zero order of diffraction.

2. The IOL of claim 1, further comprising a third diffractive region having a power of different magnitude than the first and the second regions, adapted to diffract substantially all light at a third selected wavelength projected therethrough into a corresponding single non-zero order of diffraction.

3. The IOL of claim 1, wherein the first region and the second region project light into corresponding first orders of diffraction.

4. The IOL of claim 1, wherein the first and second regions are disposed concentrically.

5. The IOL of claim 1, wherein the IOL comprises one or more haptics.

6. The IOL of claim 1, wherein the IOL is made of a material comprising at least one of silicone, PMMA, foldable hydrophilic acrylic and foldable hydrophobic acrylic.

7. The IOL of claim 4, further comprising a third region that is concentric with the first and second regions, wherein the first region, the second region, and the third region form alternating regions of near and far focus.

8. The IOL of claim 1, wherein the first selected wavelength and the second selected wavelength are at substantially a same wavelength in the visible spectrum.

9. The IOL of claim 1, wherein the first selected wavelength and the second selected wavelength are at a same wavelength in the visible spectrum.

10. The IOL of claim 1, wherein the same wavelength is approximately 555 nm.

11. A method, comprising:
projecting light of a selected wavelength onto a first diffractive region and a second diffractive region of an IOL, the first region having a first diffractive optical power and the second region having a second diffractive optical power of a different magnitude than the first diffractive optical power;
diffracting substantially all light of a first selected wavelength projected onto the first region into a single non-zero order of diffraction; and
diffracting substantially all light of a second selected wavelength projected onto the second region into a single non-zero order of diffraction.

12. The method of claim 11, wherein the first selected wavelength and the second selected wavelength are at substantially a same wavelength in the visible spectrum.

13. The method of claim 11, wherein the first selected wavelength and the second selected wavelength are at a same wavelength in the visible spectrum.

14. The method of claim 13, wherein the same wavelength is approximately 555 nm.
